SEQUENCE LISTING (14 sequences)

SEQ ID NO: 1 - DNA, 95 bp, Human adenovirus type 1
gtttttttgtggaacttgacaagctgattctaaaatttatatggaggagaaaagattcaagaatagccaagacattcctgaagaagaagaacaag

SEQ ID NO: 2 - DNA, 341 bp, Human adenovirus type 1
gtaagagccctcccgccactccaaagccttattgccccatccctcaattccctccaccccttccacttctcaggtactagttaatgattggcgtatagacaagaatcatggcatgcctcttgttgcacccacttaacaacatggcgttgccttttgttgcaccttagtggcttctggaaataacgtaaaagccaaaggctttctcctaatgagctaggaacagacatgtccttgcccagctgggattctgtctgcccagggcctgaggtggtgggagcaatgcaaggagagggagaggacaaatgatattggctagccataagccgctattcttcttacag

SEQ ID NO: 3 - protein, 8 aa, Homo sapiens
VFLWNLTS

SEQ ID NO: 4 - protein, 26 aa, Homo sapiens
CKSPPATPKPYCPIPQFPPPLPLLRY

SEQ ID NO: 5 - DNA, 30 bp, Homo sapiens
agcatggattcctggttcattcttgttctg

SEQ ID NO: 6 - DNA, 28 bp, Homo sapiens
ctctacagacacccgaatattcccatag

SEQ ID NO: 7 - DNA, 27 bp, Homo sapiens
agtactggccagaccaaggctgcggac

SEQ ID NO: 8 - DNA, 27 bp, Homo sapiens
cgcttacttgaagttggcataatctga

SEQ ID NO: 9 - DNA, 957 bp, Homo sapiens
agcatggattcctggttcattcttgttctgctcggcagtggtctgatatgtgtcagtgccaacaatgctaccacagttgcaccttctgtaggaattacaagattaattaactcatcaacggcagaaccagttaaagaagaggccaaaacttcaaatccaacttcttcactaacttctctttctgtggcaccaacattcagcccaaatataactctgggacccacctatttaaccactgtcaattcttcagactctgacaatgggaccacaagaacagcaagcaccaattctataggcattacaatttcaccaaatggaacgtggcttccagataaccagttcacggatgccagaacagaaccctgggaggggaattccagcaccgcagcaaccactccagaaactttccctccttcagatgagacaccaattattgcggtgatggtggccctgtcctctctgctagtgatcgtgtttattatcatagttttgtacatgttaaggtttaagaaatacaagcaagctgggagccattccaattctttccgcttatccaacggccgcactgaggatgtggagccccagagtgtgccacttctggccagatccccaagcaccaacaggaaatacccacccctgcccgtggacaagctggaagaggaaattaaccggagaatggcagacgtttttttgtggaacttgacaagctgattctaaaatttatatggaggagaaaagattcaagaatagccaagacattcctgaagaagaagaacaaggagcaccgtcctggagcgtgtgaaagcagaggggattttggatgtcttccagactgtcaagagcctgcggctacagaggccacacatggtccagacactggaacagtatgagttctgctacaaggtggtgcaggagtatattgatgcattctcagattatgccaacttcaagtaagcg

SEQ ID NO: 10 - DNA, 1965 bp, Homo sapiens
agcatggattcctggttcattcttgttctgctcggcagtggtctgatatgtgtcagtgccaacaatgctaccacagttgcaccttctgtaggaattacaagattaattaactcatcaacggcagaaccagttaaagaagaggccaaaacttcaaatccaacttcttcactaacttctctttctgtggcaccaacattcagcccaaatataactctgggacccacctatttaaccactgtcaattcttcagactctgacaatgggaccacaagaacagcaagcaccaattctataggcattacaatttcaccaaatggaacgtggcttccagataaccagttcacggatgccagaacagaaccctgggaggggaattccagcaccgcagcaaccactccagaaactttccctccttcagatgagacaccaattattgcggtgatggtggccctgtcctctctgctagtgatcgtgtttattatcatagttttgtacatgttaaggtttaagaaatacaagcaagctgggagccattccaattctttccgcttatccaacggccgcactgaggatgtggagccccagagtgtgccacttctggccagatccccaagcaccaacaggaaatacccacccctgcccgtggacaagctggaagaggaaattaaccggagaatggcagacgacaataagctcttcagggaggaattcaacgctctccctgcatgtcctatccaggccacctgtgaggctgcttccaaggaggaaaacaaggaaaaaaatcgatatgtaaacatcttgccttatgaccactctagagtccacctgacaccggttgaaggggttccagattctgattacatcaatgcttcattcatcaacggctaccaagaaaagaacaaattcattgctgcacaaggaccaaaagaagaaacggtgaatgatttctggcggatgatctgggaacaaaacacagccaccatcgtcatggttaccaacctgaaggagagaaaggagatgcagtatgtcttcatataccaagcccttctggagcattatctctatggagatacagaactggaagtgacctctctagaaacccacctgcagaaaatttacaacaaaatcccagggaccagcaacaatggattagaggaggagtttaagaagttaacatcaatcaaaatccagaatgacaagatgcggactggaaaccttccagccaacatgaagaagaaccgtgttttacagatcattccatatgaattcaacagagtgatcattccagttaagcggggcgaagagaatacagactatgtgaacgcatcctttattgatggctaccggcagaaggactcctatatcgccagccagggccctcttctccacacaattgaggacttctggcgaatgatctgggagtggaaatcctgctctatcgtgatgctaacagaactggaggagagaggccaggagaagtgtgcccagtactggccatctgatggactggtgtcctatggagatattacagtggaactgaagaaggaggaggaatgtgagagctacaccgtccgagacctcctggtcaccaacaccagggagaataagagccggcagatccggcagttccacttccatggctggcctgaagtgggcatccccagtgacggaaagggcatgatcagcatcatcgccgccgtgcagaagcagcagcagcagtcagggaaccaccccatcaccgtgcactgcagcgccggggcaggaaggacggggaccttctgtgccctgagcaccgtcctggagcgtgtgaaagcagaggggattttggatgtcttccagactgtcaagagcctgcggctacagaggccacacatggtccagacactggaacagtatgagttctgctacaaggtggtgcaggagtatattgatgcattctcagattatgccaacttcaagtaagcg

SEQ ID NO: 11 - DNA, 2305 bp, Homo sapiens
agcatggattcctggttcattcttgttctgctcggcagtggtctgatatgtgtcagtgccaacaatgctaccacagttgcaccttctgtaggaattacaagattaattaactcatcaacggcagaaccagttaaagaagaggccaaaacttcaaatccaacttcttcactaacttctctttctgtggcaccaacattcagcccaaatataactctgggacccacctatttaaccactgtcaattcttcagactctgacaatgggaccacaagaacagcaagcaccaattctataggcattacaatttcaccaaatggaacgtggcttccagataaccagttcacggatgccagaacagaaccctgggaggggaattccagcaccgcagcaaccactccagaaactttccctccttcagatgagacaccaattattgcggtgatggtggccctgtcctctctgctagtgatcgtgtttattatcatagttttgtacatgttaaggtttaagaaatacaagcaagctgggagccattccaattctttccgcttatccaacggccgcactgaggatgtggagccccagagtgtgccacttctggccagatccccaagcaccaacaggaaatacccacccctgcccgtggacaagctggaagaggaaattaaccggagaatggcagacgacaataagctcttcagggaggaattcaacgctctccctgcatgtcctatccaggccacctgtgaggctgcttccaaggaggaaaacaaggaaaaaaatcgatatgtaaacatcttgccttatgaccactctagagtccacctgacaccggttgaaggggttccagattctgattacatcaatgcttcattcatcaacggctaccaagaaaagaacaaattcattgctgcacaaggaccaaaagaagaaacggtgaatgatttctggcggatgatctgggaacaaaacacagccaccatcgtcatggttaccaacctgaaggagagaaaggagatgcagtatgtcttcatataccaagcccttctggagcattatctctatggagatacagaactggaagtgacctctctagaaacccacctgcagaaaatttacaacaaaatcccagggaccagcaacaatggattagaggaggagtttaagaagttaacatcaatcaaaatccagaatgacaagatgcggactggaaaccttccagccaacatgaagaagaaccgtgttttacagatcattccatgtaagagccctcccgccactccaaagccttattgccccatccctcaattccctccaccccttccatttctcaggtactagttaatgattggcgtatagacaagaatcatggcattgcctcttgttgcacccacttaacaacatggcgttgccttttgttgcaccttagtggcttctggaaataacgtaaaagccaaaggctttctccctaatgagctaggaacagacatgtccttgcccagctgggattctgtctgcccagggcctgaggtgggagcaatgcaaggagagggagaggacaaatgatattggctagccataagccgctattcttcttacagatgaattcaacagagtgatcattccagttaagcggggcgaagagaatacagactatgtgaacgcatcctttattgatggctaccggcagaaggactcctatatcgccagccagggccctcttctccacacaattgaggacttctggcgaatgatctgggagtggaaatcctgctctatcgtgatgctaacagaactggaggagagaggccaggagaagtgtgcccagtactggccatctgatggactggtgtcctatggagatattacagtggaactgaagaaggaggaggaatgtgagagctacaccgtccgagacctcctggtcaccaacaccagggagaataagagccggcagatccggcagttccacttccatggctggcctgaagtgggcatccccagtgacggaaagggcatgatcagcatcatcgccgccgtgcagaagcagcagcagcagtcagggaaccaccccatcaccgtgcactgcagcgccggggcaggaaggacggggaccttctgtgccctgagcaccgtcctggagcgtgtgaaagcagaggggattttggatgtcttccagactgtcaagagcctgcggctacagaggccacacatggtccagacactggaacagtatgagttctgctacaaggtggtgcaggagtatattgatgcattctcagattatgccaacttcaagtaagcg

SEQ ID NO: 12 - protein, 245 aa, Homo sapiens
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSDETPIIAVMVALSSLLVIVFIIIVLYMLRFKKYKQAGSHSNSFRLSNGRTEDVEPQSVPLLARSPSTNRKYPPLPVDKLEEEINRRMADDNKLFREEFNVFLWNLTS

SEQ ID NO: 13 - protein, 652 aa, Homo sapiens
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSDETPIIAVMVALSSLLVIVFIIIVLYMLRFKKYKQAGSHSNSFRLSNGRTEDVEPQSVPLLARSPSTNRKYPPLPVDKLEEEINRRMADDNKLFREEFNALPACPIQATCEAASKEENKEKNRYVNILPYDHSRVHLTPVEGVPDSDYINASFINGYQEKNKFIAAQGPKEETVNDFWRMIWEQNTATIVMVTNLKERKEMQYVFIYQALLEHYLYGDTELEVTSLETHLQKIYNKIPGTSNNGLEEEFKKLTSIKIQNDKMRTGNLPANMKKNRVLQIIPYEFNRVIIPVKRGEENTDYVNASFIDGYRQKDSYIASQGPLLHTIEDFWRMIWEWKSCSIVMLTELEERGQEKCAQYWPSDGLVSYGDITVELKKEEECESYTVRDLLVTNTRENKSRQIRQFHFHGWPEVGIPSDGKGMISIIAAVQKQQQQSGNHPITVHCSAGAGRTGTFCALSTVLERVKAEGILDVFQTVKSLRLQRPHMVQTLEQYEFCYKVVQEYIDAFSDYANFK

SEQ ID NO: 14 - protein, 445 aa, Homo sapiens
MDSWFILVLLGSGLICVSANNATTVAPSVGITRLINSSTAEPVKEEAKTSNPTSSLTSLSVAPTFSPNITLGPTYLTTVNSSDSDNGTTRTASTNSIGITISPNGTWLPDNQFTDARTEPWEGNSSTAATTPETFPPSDETPIIAVMVALSSLLVIVFIIIVLYMLRFKKYKQAGSHSNSFRLSNGRTEDVEPQSVPLLARSPSTNRKYPPLPVDKLEEEINRRMADDNKLFREEFNALPACPIQATCEAASKEENKEKNRYVNILPYDHSRVHLTPVEGVPDSDYINASFINGYQEKNKFIAAQGPKEETVNDFWRMIWEQNTATIVMVTNLKERKEMQYVFIYQALLEHYLYGDTELEVTSLETHLQKIYNKIPGTSNNGLEEEFKKLTSIKIQNDKMRTGNLPANMKKNRVLQIIPCKSPPATPKPYCPIPQFPPPLPFLRY

(19) United States
(12) Patent Application Publication
(10) Pub. No.: US 2014/0120108 A1
(43) Pub. Date: May 1, 2014

(54) MUTANT PTP ALPHA GENE GROUP IN MALIGNANT TUMORS AND PRODUCTION METHOD

(71) Applicant: SHANGHAI SIGNAL BIOTECH CO., LTD. (CN), Shanghai (CN)

(72) Inventor: Zhiwei Pan, Shanghai (CN)

(73) Assignee: SHANGHAI SIGNAL BIOTECH CO., LTD. (CN), Shanghai (CN)

(21) Appl. No.: 14/140,455

(22) Filed: Dec. 24, 2013

(30) Foreign Application Priority Data
Dec. 29, 2009 (CN) 200910247437.4
Nov. 25, 2010 (CN) PCT/CN2010/079111

(51) Int. Cl. C12Q 1/68

(52) U.S. Cl. CPC C12Q 1/6886
USPC 424/139.1; 435/6.12; 435/7.4; 514/44 A; 536/23.2

(57) ABSTRACT

A group of mutant PTPαgenes in malignant tumor are provided, which are ΔPTPα245, ΔPTPα652 and ΔPTPα445 respectively. The mutation includes insertion of 95 new nucleotides after nucleotide at position 711, deletion of nucleotides at position 1015-1437, and deletion of nucleotides at position 1015-1437 accompanied by insertion of 340 nucleotides after coding exon at position 1681 and fusion of 26 new amino acids at C-terminal. The group of mutant PTPαgenes in different types of malignant tumor disclosed in the present application have not been reported all over the world so far. The detection method of using PTPαmutant genes is useful in exactly diagnosing malignant tumor, developing new anti-tumor drugs, and targeted treatment at molecular pathologic level.

This application is a continuation application of U.S. application Ser. No. 13/383,065 entitled “MUTANT PTP ALPHA GENE GROUP IN MALIGNANT TUMORS AND PRODUCTION METHOD” filed on Jan. 09, 2012 by Zhiwei Pan, which was the U.S. national phase of International Application No. PCT/CN2010/079111 Filed on Nov. 25, 2010 which designated the U.S. and claims priority to Chinese Application Nos. 200910247437.4 filed on Dec. 29, 2009, the entire contents of each of which are hereby incorporated by reference.

### TECHNICAL FIELD

The present invention relates to a mutant PTP a gene group in malignant tumors.

### BACKGROUND ART

Protein Tyrosine Kinases (PTKs) and Protein Tyrosine Phosphatases (PTPs) respectively represent two enzyme families, and both maintain the vital activity of normal cells through positive and negative regulation of phosphorylation and dephosphorylation, e.g. systematic growth, development, differentiation and apoptosis. The appearance of malignant tumor is typically caused by the fact that such normal balance regulation becomes incontrollable, for example, the mutation of one or several key enzymatic genes in the two enzyme families or the activation of enzymatic activity by any factor is essentially responsible for the induction of malignant tumor.

PTP α (Protein Tyrosine Phosphatase α) is a member of the protein tyrosine phosphatase family, consists of 793 amino acids, has the molecular weight of 130 KDa, and can specifically catalyze phosphoric acid modified on tyrosine residue to be dephosphorylated. Signal transduction dominated by Src tyrosine phosphokinase is regulated by dephosphorylating tyrosine phosphokinase-catalyzed substrate of proto-oncogene Src family, in order to maintain normal cell growth and mitosis. PTP α is also receptor-type transmembrane protein tyrosine phosphatase that participates not only in signal channels for Epidermal Growth Factor Receptor (EGFR) and Insulin Receptor (IR), but also in the regulation of cell migration, and that includes the function of inhibiting the apoptosis of tumor cells.

Since PTP α gene was cloned in 1990, relevant mutant PTP α genes in malignant tumors have not been reported domestically and overseas yet and high efficiency technology for detecting the mutant of PTP α gene has not been invented.

### SUMMARY OF THE INVENTION

The technical problem to be solved by the present invention is to provide a mutant PTP α gene in malignant tumors.

The second technical problem to be solved by the present invention is to provide the use of the mutant PTP α gene in diagnosing malignant tumors and in targeted therapy of malignant tumors.

In order to solve the first problem above, the present invention provides three mutant PTP α genes as below: ΔPTP α 245 as shown in SEQ ID NO: 9 which encodes a peptide as shown in SEQ ID NO: 12 ΔPTP α 652 as shown in SEQ ID NO: 10 which encodes a peptide as shown in SEQ ID NO: 13 and ΔPTP α 445 as shown in SEQ ID NO: 11 which encodes a peptide as shown in SEQ ID NO: 14.

A mutant PTP α gene in malignant tumors is characterized in that the gene is ΔPTP α 245 as shown in SEQ ID NO: 9, wild type PTP α gene has the length of 2379 bp with 20 encoding exons in total as follows: exon 1: 1-73 bp; exon 2: 74-415 bp; exon 3: 416-500 bp; exon 4:501-574 bp; exons: 575-711 bp; exon 6: 712-802 bp; exon 7: 803-879 bp; exon 8: 880-916 bp; exon 9: 917-1014 bp; exon 10: 1015-1134 bp; exon 11: 1035-1301 bp; exon 12: 1302-1437 bp: exon 13: 1438-1587 bp; exon 14: 1588-1681 bp; exon 15: 1682-1758 bp; exon 16: 1759-1893 bp; exon 17:1894-2019 bp; exon 18: 2020-2171 bp; exon 19: 2172-2307 bp; exon 20: 2308-2379 bp (Kapp, K., et al., 2007, Extracellular domain splice variants of a transforming protein tyrosine phosphatase a mutant differentially activate Src-kinase dependent focus formation. Genes to Cells 12: 63-73), the mutant location of the mutant PTP α gene: 95 new nucleotide segments are inserted behind the 711^{th} nucleotide; gene modification: partial deletion of the 6^{th} to the 20^{th} encoding exons is initiated; protein modification: protein contains 245 amino acids with 8 new amino acids therein located at c-end.

The 95 new nucleotide sequences are shown as SEQ ID NO: 1.

The 8 new amino acid sequences are shown as follows: —V F L W N L T S—, as shown in SEQ ID NO:3.

A mutant PTP α gene in malignant tumors is characterized in that the gene is ΔPTP α 652 as shown in SEQ ID NO: 10, wild type PTP α gene has the length of 2379 bp with 20 encoding exons in total as follows: exon 1: 1-73 bp; exon 2: 74-415 bp; exon 3: 416-500 bp; exon 4:501-574 bp; exon 5: 575-711 bp; exon 6: 712-802 bp; exon 7: 803-879 bp; exon 8:880-916bp; exon 9: 917-1014bp; exon 10: 1015-1134bp; exon 11: 1035-1301 bp; exon 12: 1302-1437 bp: exon 13: 1438-1587 bp: exon 14: 1588-1681 bp; exon 15: 1682-1758 bp; exon 16: 1759-1893 bp; exon 17:1894-2019 bp; exon 18: 2020-2171 bp; exon 19: 2172-2307 bp; exon 20: 2308-2379 bp, the mutant location of the gene: deletion of the 1015^{th} to 1437^{th} nucleotides; gene modification: deletion of the 10^{th}, 11^{th} and 12^{th} encoding exons is initiated; protein modification: protein contains 652 amino acids;

E in the wild type PTP α gene represents exon;

The ΔPTP α 652 gene has the deletion of the 10^{th}, 11^{th} and 12^{th} exons and the deletion of 423 nucleotides.

A mutant PTP α gene in malignant tumors is characterized in that the gene is ΔPTP α 445 as shown in SEQ ID NO: 11, wild type PTP α gene has the length of 2379 bp with 20 encoding exons in total as follows: exon 1: 1-73 bp; exon 2: 74-415 bp; exon 3: 416-500 bp; exon 4:501-574 bp; exon 5: 575-711 bp; exon 6: 712-802 bp; exon 7: 803-879 bp; exon 8:880-916 bp; exon 9: 917-1014 bp; exon 10: 1015-1134 bp; exon 11: 1035-1301 bp; exon 12: 1302-1437 bp: exon 13: 1438-1587 bp: exon 14: 1588-1681 bp; exon 15: 1682-1758 bp; exon 16: 1759-1893 bp; exon 17:1894-2019 bp; exon 18: 2020-2171 bp; exon 19: 2172-2307 bp; exon 20: 2308-2379 bp, the mutant location of the gene: deletion of the 1015^{th} to 1437^{th} nucleotides, accompanied with the insertion of 340 nucleotides behind the 1681^{th} encoding exon and the fusion of 26 new amino acids at c-end; gene modification: deletion of the 15^{th} to 20^{th} encoding exons is initiated; protein modification: protein contains 445 amino acids;

The 341 nucleotides are intact 14^{th} intron sequence, which is shown as SEQ ID NO: 2.

The exons of the wild type PTP α gene are shown as FIG. 1, and E represents exon.

The exons of the ΔPTP α 445 gene are shown as FIG. 4.

In order to solve the second problem above, it is found from the PTP α gene sequencing and analysis on samples of 38 various tumor tissues that a part of PTP α genes is mutant, shown as Table 1 and Table 2.

TABLE 1

| | Gene | Type of | Case | Protein |
|---|---|---|---|---|
| Mutant Location | Modification | Tumor | Number | Modification |
| Insertion of 95 | Deletion of the | Intestinal | 2 | ΔPTP α 245 |
| nucleotides, | 6^{th} to 20^{th} exons | cancer | | |
| initiation of | | | | |
| deleted sequence | | | | |
| 712 to 2379 | | | | |
| Insertion of 95 | Deletion of the | Mammary | 1 | ΔPTP α 245 |
| nucleotides, | 6^{th} to 20^{th} exons | cancer | | |
| initiation of | | | | |
| deleted sequence | | | | |
| 712 to 2379 | | | | |
| Insertion of 95 | Deletion of the | Liver | 1 | ΔPTP α 245 |
| nucleotides, | 6^{th} to 20^{th} exons | cancer | | |
| initiation of | | | | |
| deleted sequence | | | | |
| 712 to 2379 | | | | |
| Deleted | Deletion of the | Mammary | 1 | ΔPTP α 652 |
| sequence 1015 | 10^{th}, 11^{th} and 12^{th} | cancer | | |
| to 1437 | exons | | | |
| Deleted | Deletion of the | Intestinal | 1 | ΔPTP α 445 |
| sequence 1015 | 10^{th}, 11^{th} and 12^{th} | cancer | | |
| to 1437, deleted | exons and the | | | |
| sequence 1682 | 15^{th} to 20^{th} exons | | | |
| to 2379 and | | | | |
| insertion of 340 | | | | |
| nucleotides | | | | |

TABLE 2

| | Type of Tumor | | | | | |
|---|---|---|---|---|---|---|
| | | Intes- | | | | Esoph- |
| | Thyroid | tinal | Liver | Mammary | Lung | agus |
| | Cancer | Cancer | Cancer | Cancer | Cancer | Cancer |
| Mutant case | 0/10 | 3/8 | 1/2 | 2/9 | 0/7 | 0/2 |
| number/ | | | | | | |
| detection | | | | | | |
| case number | | | | | | |
| Mutation | 0% | 38% | 50% | 33% | 0% | 0% |
| percentage | | | | | | |

The RT-PCR results of different types of tumor samples are shown in FIG. 5.

The 26 new amino acid sequences are as follows: —C KS P P A T P K P Y C P I P Q F P P P L P L L R Y—, as shown in SEQ ID NO: 4.

A production method of a mutant PTP α gene group in malignant tumors is characterized in that the method comprises the following steps of:

### A. Cloning of PTP α Mutant Gene

(1) Extraction of Total RNA

A patient's tumor tissue resulting from surgical excision is cut into pieces and RNA is extracted by 1 ml of guanidine isothiocyanate/phenol solution (Verhofstede et al. 1996 Isolation of HIV-1 RNA from plasma: evaluation of eight difference extraction methods. J. of Virol. Method 60: 155-159; Chomczynski P & Sacchi N, 1987 Single-step method of RNA isolation by acid guanidinium thiocynate-phenol-chroloform extraction. Anal. Biochem. 162: 156-159), the addition of 0.2 ml of chloroform is followed by violent shaking for 15 seconds, placement for 10 minutes at room temperature and centrifugation for 15 minutes at the speed of 15000 rpm at 4° C., supernatant is sucked and added with 0.5 ml of isopropanol for homogeneous mixing, the mixture is put on a standing for 10 minutes and then centrifuged for 10 minutes at the speed of 15000 rpm, the supernatant is removed, precipitates are washed with 75% ethanol and then dissolved in 20 ul of DEPC-H20. After 2 ul of the resultant solution is diluted, absorbance is determined by ultraviolet spectrophotometer.

(2) RT-PCR

1 ug of the above RNA is taken by using a reverse transcription kit, 1 μl of random primer and 1 μl of dNTP are added, 10 μl is complemented by DEPC-H₂O, the RNA is put at 65° C. for 5 minutes and then placed in ice bath immediately. The addition of 10 μl of cDNA synthetic mixed liquid is followed by placement for 10 minutes at 25° C., placement for 50 minutes at 50° C., placement for 5 minutes at 85° C. and placement in ice bath immediately, the addition of 1 μl of RNaseH is followed by placement in 37° C. water bath for 20 minutes, and the cDNA is preserved at −20° C.

The amplification of PTP α gene by Polymerase Chain Reaction(PCR) of sample DNA comprises two parts: the first part of PTP α 1 forward primer sequence: 5′-AGCATGGATTCCTGGTTCATTCTTGTTCTG-3′, which is shown in SEQ ID NO: 5, reverse primer sequence: 5′-CTCTACAGACACCCGAATATTCCCATAG-3′, which is shown in SEQ ID NO: 6, the second part of PTP α 2 forward primer sequence: 5′-AGTACTGGCCAGACCAAGGCTGCGGAC-3′, which is shown in SEQ ID NO: 7, and reverse primer sequence: 5′ -CGCTTACTTGAAGTTGGCATAATCTGA-3′, which is shown in SEQ ID NO: 8. The amplification system is as follows: 5 μl of 10× buffer solution, 2 μl of dNTP, 0.5 μl of 10 μmol/L forward primer, 0.5 μl of 10 μmol/L reverse primer, 3 μl of cDNA, and double distilled water complementary to volume 48 μl. After 95° C. deactivation is performed for 5 minutes, 1 U (diluted to 1 U/2 μl prior to application) Taq DNA Polymerase (Markoulatos P. et al. 2003 Multiplex PCR: Rapid DNA cycling in a conventional thermal cycler. J. Clin. Lab. Anal. 17: 108-112) is added. The amplification conditions include 40 seconds at 90° C., 40 seconds at 55° C., 120 seconds at 68° C. and 30 cycles in total.

### B. Purification of PCR Products for Sequencing

(1) Ligation and Transformation of PCR Product

The above PCR (Polymerase Chain Reaction) product is subjected to electrophoretic separation with 1% of agarose gel, and then the right PCR product is ligated to a TA vector (Tirado G & Kumar A. 2006 Evolution of SIV envelope in morphine-dependent Rhesus Macaques with Rapid Disease progression. AIDS Research and Human Retroviruses 22: 114-119) with the length of 3.9 kb and transformed into Escherichia coli cells. The specific steps are as follows: 1 ul of saline solution and lul of TA vector are added to 4 μl of the PCR product, they are lightly and homogeneously mixed, and then, the mixture is on standing for 5 minutes at room temperature and placed for 10 minutes at 30° C. to wait for ligation. Afterwards, 2 μl of the mixture is taken out and added to E. coli (Escherichia coli) solution, they are lightly and homogeneously mixed, the mixture is placed on ice for 10 minutes to wait for conversion and then placed in water bath at 42° C. for 30 seconds in order to implement reversed heat shock, and after that, the mixture is placed in ice bath immediately. 250 μl of S.O.C. culture solution is added at room temperature, and after the lid is fastened, 1-hour shaking on a constant temperature shaking table at 37° C. is performed for recovery.

(2) Screening, Identification and Determination

100 μl of converted bacterial liquid is dropwise added to 1.5% LB agar plate containing 100 μg/ml of ampicillin, then 40 ul of X-gal is added, uniform coating is completed by a glass rod immediately, then the plate is placed in a constant temperature incubator at 37° C. for incubation for 18 hours, white colony is picked out and transferred to a 3 ml LB liquid culture medium, shaking culture is implemented over night by the constant temperature shaking table at 37° C., and a plasmid miniprep kit of Geneaid is used for extracting bacterial plasmid. The bacterial liquid is centrifuged for 2 minutes at the speed of 6000 rpm at first and supernatant is then removed, precipitates are added with 200 μl of RNaseA-containing solution I, bacteria is re-suspended, 200 ul of solution II is added and the solution is reversed lightly, mixed homogeneously and placed for 5 minutes for the purpose of cell lysis, then 300 μl of solution III is added, the solution is reversed lightly, mixed homogeneously and centrifuged for 5 minutes, supernatant is transferred to a centrifugal column for centrifugation for 30 seconds at the speed of 10000 rpm, washing buffer solution containing ethanol is then added, centrifugation is implemented for 30 seconds at the speed of 10000 rpm, the buffer solution is completely removed, 50 μl of 110 mmol/LTris-HCL buffer solution (pH 8.5) is added to dissolve DNA, followed by standing for 2 minutes and then centrifugation for 2 minutes, and effluent liquid, which is bacterial plasmid DNA, is collected. 5 μl of DNA is added with 2 μl of 10× buffer solution and 10 U restriction endonuclease EcoR I, 20 ul volume is complemented by double distilled water, enzyme digestion is implemented for 2 hours in water bath at 37° C., electrophoretic separation is implemented with 1% of agarose gel, external segments are indeed present on plasmid in accordance with the identification, and DNA sequencing is implemented.

The present invention has the advantages that: a mutant PTP α gene group in malignant tumors, disclosed by the present invention, has not been reported domestically and overseas so far, and the application of the method for detecting mutant PTP α genes can bring direct guidance significance to the accurate diagnosis of malignant tumors in the aspect of molecular pathologic level, the development of new antitumor drugs and the targeted therapy.

### BRIEF DESCRIPTION OF THE DRAWINGS

FIG. 1: 20 exons of wild type PTP α gene, E represents exon.

FIG. 2: Exons of ΔPTP α 245 as shown in SEQ ID NO: 9, 95 new nucleotide segments are inserted behind the 7111^{th} nucleotide, and partial deletion of the 6^{th} to the 20^{th} encoding exons is initiated.

FIG. 3: Exons of ΔPTP α 652 as shown in SEQ ID NO: 10, deletion of the 10^{th}, 11^{th} and 12^{th} exons.

FIG. 4: Exons of ΔPTP α 445 as shown in SEQ ID NO: 11, deletion of the 10^{th}, 11^{th} and 12^{th} exons, accompanied by the insertion of 340 nucleotides behind the 1681^{th} encoding exon.

FIG. 5: The RT-PCR results of different types of tumor samples, M represents 0.5 kb DNA Ladder; 1. normal mammary tissue; 2, mammary cancer 62; 3, normal liver tissue; 4, liver cancer; 5, normal colonic tissue; 6. intestinal cancer; ΔPTP α 245 as shown in SEQ ID NO: 9 is mutant gene; PTP α is wild type gene.

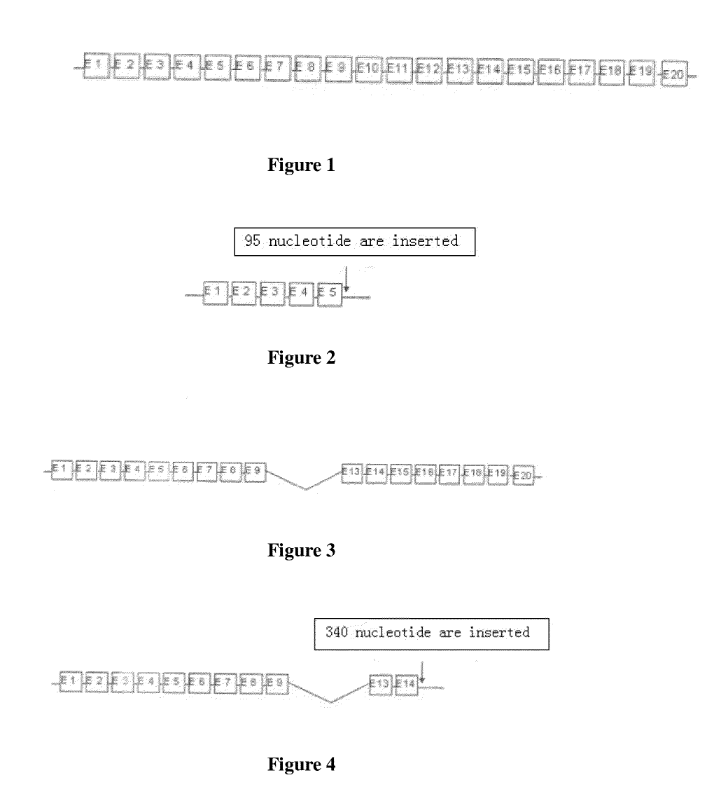

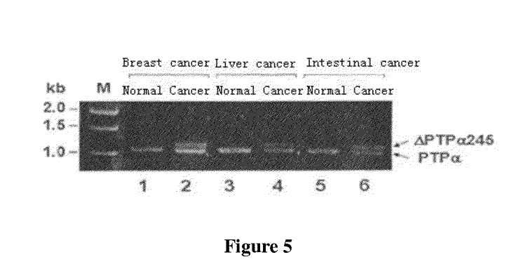

### DETAILED DESCRIPTION OF THE EMBODIMENTS

Detailed description is made below to the embodiments of technical proposal provided by the invention with reference to the drawings.

### Embodiment

I. Cloning of PTP α Mutant Gene

(1) Extraction of Total RNA

A patient's tumor tissue resulting from surgical excision is cut into pieces and RNA is extracted by 1 ml of guanidine isothiocyanate/phenol solution, the addition of 0.2 ml of chloroform is followed by violent shaking for 15 seconds, placement for 10 minutes at room temperature and centrifugation for 15 minutes at the speed of 15000 rpm at 4° C., supernatant is sucked and added with 0.5 ml of isopropanol for homogeneous mixing, the mixture is put on a standing for 10 minutes and then centrifuged for 10 minutes at the speed of 15000 rpm, the supernatant is removed, precipitates are washed with 75% ethanol and then dissolved in 20 ul of DEPC-H20. After 2 μl of the resultant solution is diluted, absorbance is determined by ultraviolet spectrophotometer.

(2) RT-PCR

1 ug of the above RNA is taken by using a reverse transcription kit, 1 μl of random primer and 1 ul of dNTP are added, 10 μl is complemented by DEPC-H20, the RNA is put at 65° C. for 5 minutes and then placed in ice bath immediately. The addition of 10 μl of cDNA synthetic mixed liquid is followed by placement for 10 minutes at 25° C., placement for 50 minutes at 50° C., placement for 5 minutes at 85° C. and placement in ice bath immediately, the addition of 1 μl of RNaseH is followed by placement in 37° C. water bath for 20 minutes, and the cDNA is preserved at −20° C.

The amplification of PTP α gene by Polymerase Chain Reaction(PCR) of sample DNA comprises two parts: the first part of PTP α 1 forward primer sequence: 5′-AGCATGGATTCCTGGTTCATTCTTGTTCTG-3′, which is shown as SEQ ID NO: 5, reverse primer sequence: 5′-CTCTACAGACACCCGAATATTCCCATAG-3′, which is shown as SEQ ID NO: 6, the second part of PTP α 2 forward primer sequence: 5′-AGTACTGGCCAGACCAAGGCTGCGGAC-3′, which is shown as SEQ ID NO: 7, and reverse primer sequence: 5′-CGCTTACTTGAAGTTGGCATAATCTGA-3′, which is shown as SEQ ID NO: 8. The amplification system is as follows: 5 μl of 10× buffer solution, 2 μl of dNTP, 0.5 μl of 10 μmol/L forward primer, 0.5 μl of 10 μmol/L reverse primer, 3 μl of cDNA, and double distilled water complementary to volume 48 μl. After 95° C. deactivation is performed for 5 minutes, 1 U (diluted to 1 U/2μl prior to application) Tag DNA Polymerase is added. The amplification conditions include 40 seconds at 90° C., 40 seconds at 55° C., 120 seconds at 68° C. and 30 cycles in total.

II. Purification of PCR Products for Sequencing

(1) Ligation and Transformation of PCR Product

The above PCR (Polymerase Chain Reaction) product is subjected to electrophoretic separation with 1% of agarose gel, and then the right PCR product is ligated to a TA vectorwith the length of 3.9 kb and transformed into Escherichia coli cells. The specific steps are as follows: 1 ul of saline solution and 1 ul of TA vector are added to 4 μl of the PCR product, they are lightly and homogeneously mixed, and then, the mixture is on standing for 5 minutes at room temperature and placed for 10 minutes at 30° C. to wait for ligation. Afterwards, 2 μl of the mixture is taken out and added to E. coli (Escherichia coli) solution, they are lightly and homogeneously mixed, the mixture is placed on ice for 10 minutes to wait for transformation and then placed in water bath at 42° C. for 30 seconds in order to implement reversed heat shock, and after that, the mixture is placed in ice bath immediately. 250 μl of S.O.C. culture solution is added at room temperature, and after the lid is fastened, 1-hour shaking on a constant temperature shaking table at 37° C. is performed for recovery.

(2) Screening, Identification and Determination

100 μl of well-converted bacterial liquid is dropwise added to 1.5% LB agar plate containing 100 μg/ml of ampicillin, then 40 ul of X-gal is added, uniform coating is completed by a glass rod immediately, then the plate is placed in a constant temperature incubator at 37° C. for incubation for 18 hours, white colony is picked out and transferred to a 3 ml LB liquid culture medium, shaking culture is implemented over night by the constant temperature shaking table at 37° C., and a plasmid miniprep kit of Geneaid is used for extracting bacterial plasmid. The bacterial liquid is centrifuged for 2 minutes at the speed of 6000 rpm at first and supernatant is then removed, precipitates are added with 200 μl of RNaseA-containing solution I, bacteria is re-suspended, 200 μl of solution II is added and the solution is reversed lightly, mixed homogeneously and placed for 5 minutes for the purpose of cell lysis, then 300 μl of solution III is added, the solution is reversed lightly, mixed homogeneously and centrifuged for 5 minutes, supernatant is transferred to a centrifugal column for centrifugation for 30 seconds at the speed of 10000 rpm, washing buffer solution containing ethanol is then added, centrifugation is implemented for 30 seconds at the speed of 10000 rpm, the buffer solution is completely removed, 50 μl of 110 mmol/LTris-HCL buffer solution (pH 8.5) is added to dissolve DNA, followed by standing for 2 minutes and then centrifugation for 2 minutes, and effluent liquid, which is bacterial plasmid DNA, is collected. 5 ul of DNA is added with 2 μl of 10× buffer solution and 10 U restriction endonuclease Eco RI, 20 μl volume is complemented by double distilled water, enzyme digestion is implemented for 2 hours in water bath at 37° C., electrophoretic separation is implemented with 1% of agarose gel, external segments are indeed present on plasmid in accordance with the identification, and DNA sequencing is implemented.

The person skilled in the art can develop a diagnostic method for malignant tumors based on the nucleic acid sequence of SEQ ID NO: 1, such as performing a polymerase chain reaction on the tissue sample of said subject for detecting said mutant nuclei acid, or detecting the absence of exons 7-19 or intron 7-19. In the same time, we can develop a new diagnostic method by detecting the peptide having the amino acid sequence of SEQ ID NO: 3, such as producing antibodies against the peptides which contain the peptide having the amino acid sequence of SEQ ID NO: 3. The malignant tumors include intestinal tumor, mammary cancer, liver cancer, lung cancer, thyroid cancer, colon cancer, skin cancer, prostate cancer and esophagus cancer.

Based on the nuclei acid sequence of SEQ ID NO: 1, we can design small interfering RNAs to specially inhibit expression of RNA whose sequence corresponded to the nuclei acid sequence of SEQ ID NO: 9. We also use antibodies against peptides containing the amino acid sequence of SEQ ID NO: 3 to inhibit the activity of a protein having the amino acid sequence of SEQ ID NO: 12.

We can develop a pharmaceutically acceptable composition to treat malignant tumors which include intestinal tumor, mammary cancer, liver cancer, lung cancer, thyroid cancer, colon cancer, skin cancer, prostate cancer and esophagus cancer. The active agent in the pharmaceutically acceptable composition can be a small interfering RNA which can specially inhibit expression of RNA whose sequence corresponded to the nuclei acid sequence of SEQ ID NO: 9, or/and an antibody against peptides containing the amino acid sequence of SEQ ID NO: 3 which can inhibit the activity of a protein having the amino acid sequence of SEQ ID NO: 12, or/and one or chemical compound(s) which can inhibit the activity of a protein having the amino acid sequence of SEQ ID NO: 12.

The person skilled in the art can develop a diagnostic method for malignant tumors based on the nucleic acid sequence of SEQ ID NO: 2, such as performing a polymerase chain reaction on the tissue sample of said subject for detecting said mutant nuclei acid. In the same time, we can develop a new diagnostic method by detecting the peptide having the amino acid sequence of SEQ ID NO: 4, such as producing antibodies against the peptides which contain the peptide having the amino acid sequence of SEQ ID NO: 4. The malignant tumors include intestinal tumor, mammary cancer, liver cancer, lung cancer, thyroid cancer, colon cancer, skin cancer, prostate cancer and esophagus cancer.

Based on the nuclei acid sequence of SEQ ID NO: 2, we can design small interfering RNAs to specially inhibit expression of RNA whose sequence corresponded to the nuclei acid sequence of SEQ ID NO: 11. We also use antibodies against peptides containing the amino acid sequence of SEQ ID NO: 4 to inhibit the activity of a protein having the amino acid sequence of SEQ ID NO: 14.

We can develop a pharmaceutically acceptable composition to treat malignant tumors which include intestinal tumor, mammary cancer, liver cancer, lung cancer, thyroid cancer, colon cancer, skin cancer, prostate cancer and esophagus cancer. The active agent in the pharmaceutically acceptable composition can be a small interfering RNA which can specially inhibit expression of RNA whose sequence corresponded to the nuclei acid sequence of SEQ ID NO: 11, or/and an antibody against peptides containing the amino acid sequence of SEQ ID NO: 4 which can inhibit the activity of a protein having the amino acid sequence of SEQ ID NO: 14, or/and one or chemical compound(s) which can inhibit the activity of a protein having the amino acid sequence of SEQ ID NO: 14.

The person skilled in the art can develop a diagnostic method for malignant tumors based on the nucleic acid sequence of SEQ ID NO: 10 and SEQ ID NO: 10 which lack exons 10-12, or based on the nucleic acid sequence of SEQ ID NO: 9 which lacks exon 7-19. The polymerase chain reaction on the tissue sample of said subject can be utilized. The malignant tumors include intestinal tumor, mammary cancer, liver cancer, lung cancer, thyroid cancer, colon cancer, skin cancer, prostate cancer and esophagus cancer.

What is described above pertains merely to the preferred embodiments of the present invention, it shall be noted that several improvements and modifications can be made by ordinary skilled in this art without departing from the principle and premise of the present invention, and these improvements and modifications shall also be considered to be within the scope of protection of the present invention.

What is claimed is:

1. An isolated cDNA having the nucleic acid sequence of SEQ ID NO: 9, said nucleic acid encoding a protein having the amino acid sequence of SEQ ID NO: 12.

2. The isolated cDNA according to claim 1, said cDNA comprises a mutant cDNA having the nucleic acid sequence of SEQ ID NO: 1 while said protein comprises a mutant polypeptide having the amino acid sequence of SEQ ID NO: 3.

3. The isolated cDNA according to claim 1, wherein the isolated cDNA is inserted in a vector.

4. The isolated cDNA according to claim 4, wherein the vector is prepared by the following steps:
a) To isolate total RNA from tumor tissue of a subject;
b) To perform a polymerase chain reaction on said total RNA with primers consisting of nucleic acids having the nucleic acid sequences of SEQ ID NO: 5, SEQ ID NO: 6, SEQ ID NO: 7 and SEQ ID NO:8;
c) To ligate a DNA product of said polymerase chain reaction into a TA vector;
d) To transform said TA vector containing said DNA product in a bacterium;
e) To identify a transformed bacterium containing a plasmid being ligated said DNA product;
f) To culture said transformed bacteria;
g) To isolate said plasmids.

5. A method for diagnosing malignant tumor in a subject, comprising detecting a mutant cDNA having the nucleic acid sequence of SEQ ID NO: 1, or/and detecting the absence of exons 7-19 or introns 7-19 of PTP alpha gene, or/and detecting a polypeptide having the amino acid sequence of SEQ ID NO: 3.

6. The method for diagnosing malignant tumor according to claim 5, wherein performing a polymerase chain reaction on the tissue sample of said subject for detecting said mutant nuclei acid.

7. The method for diagnosing malignant tumor according to claim 5, wherein an antibody is utilized for detecting said polypeptide, said antibody specifically interacts with the mutant polypeptide of claim 2.

8. The method for diagnosing malignant tumor according to claim 5, wherein said malignant tumor is selected from a group of intestinal tumor, mammary cancer, liver cancer, lung cancer, thyroid cancer, colon cancer, skin cancer, prostate cancer and esophagus cancer.

9. The method for diagnosing malignant tumor according to claim 6, the primers of said polymerase chain reaction can be SEQ ID NO: 5, SEQ ID NO: 6, SEQ ID NO: 7 and SEQ ID NO: 8.

10. The method for diagnosing malignant tumor according to claim 6, the DNA templates of said polymerase chain reaction can be either cDNA or genomic DNA from said subject.

11. A method for treating malignant tumor in a subject, comprising destroying said malignant tumor by inhibiting expression of RNA whose sequence corresponded to the nuclei acid sequence of SEQ ID NO: 9, or/and by inhibiting the activity of a protein having the amino acid sequence of SEQ ID NO: 12.

12. The method for treating malignant tumor according to claim 11, wherein the inhibition for the expression of RNA by utilizing a small interfering RNA having a nuclei acid sequence being designed by referring the nuclei acid sequence of SEQ ID NO: 1.

13. The method for treating malignant tumor according to claim 11, wherein a pharmaceutically acceptable composition is applied.

14. The method for treating malignant tumor according to claim 11, wherein said malignant tumor is selected from a group of intestinal tumor, mammary cancer, liver cancer, lung cancer, thyroid cancer, colon cancer, skin cancer, prostate cancer and esophagus cancer.

15. The method for treating malignant tumor according to claim 13, wherein the pharmaceutically acceptable composition contains an acceptable chemical compound.

16. The method for treating malignant tumor according to claim 13, wherein the pharmaceutically acceptable composition contains an antibody specifically interacted with the mutant polypeptide of claim 2.

17. The method for treating malignant tumor according to claim 13, wherein the pharmaceutically acceptable composition includes the small interfering RNA having a nuclei acid sequence being designed by referring the nuclei acid sequence of SEQ ID NO: 1.
